(19) United States
(12) Patent Application Publication
(10) Pub. No.: US 2011/0230790 A1
(43) Pub. Date: Sep. 22, 2011

(54) Method and system for sleep monitoring, regulation and planning

(76) Inventor: Valeriy Kozlov, Lviv (UA)

(21) Appl. No.: 12/748,403

(22) Filed: Mar. 27, 2010

(30) Foreign Application Priority Data
Mar. 16, 2010 (UA) 201003014

(51) Int. Cl. A61B 5/11; A61B 5/00

(52) U.S. Cl. 600/595; 600/300

(57) ABSTRACT

A method for operating a sleep phase actigraphy synchronized alarm clock that communicates with a remote sleep database, such as an internet server database, and compares user physiological parameters, sleep settings, and actigraphy data with a large database that may include data collected from a large number of other users with similar physiological parameters, sleep settings, and actigraphy data. The remote server may use “black box” analysis approach by running supervised learning algorithms to analyze the database, producing sleep phase correction data which can be uploaded to the alarm clock, and be used by the alarm clock to further improve its REM sleep phase prediction accuracy.

### FIELD OF THE INVENTION

The invention can be used in medical applications, as well as for physiological human sleep monitoring, regulation and planning in a home environment.

### BACKGROUND OF THE INVENTION

Humans spend around 30% of their lives sleeping. Many physiological processes underlying well-being are closely connected with sleep, and a decrease in sleep quality affects well-being. Thus, there is a need for improved home environment sleep monitoring, regulation and planning systems to improve the quality of sleep.

A number of prior sleep monitoring, regulation, and planning systems and methods exist. These are primarily based on measurements of human biometric data during sleep, and this biometric data can be used to detect the phase of the user's sleep cycle. As a rule, these systems and methods have been used for medical purposes to treat sleep disorders and other illnesses related with sleep and its characteristics.

These systems and methods can be also used as natural alarm clock algorithms for everyday use.

In certain sleep phases, a human body is more prepared for awakening than in other sleep phases. For instance, a human body is better prepared for awakening during REM (Rapid Eye Movement) sleep. During REM sleep pulse and heart rate speed up, and brain temperature and blood pressure increase resulting in increase of brain activity.

If a person is awakened at the end of REM sleep, as a rule they feel better than after waking up from any other sleep phase. By contrast, if a person is awakened during a different sleep phase, such as the deep sleep phase, the results are not as favorable. In the deep sleep phase the body (and the brain as well) is completely relaxed (pulse rate becomes more stable comparing to REM phase, blood pressure falls and brain temperature decreases), thus awakening from a deep sleep is uncomfortable, and as a result, a person awakening from deep sleep can feel groggy and unrested.

One method to detect sleep phases is by measurement of body movements during sleep (actigraphy). Using actigraphy analysis of body motions, it is possible to determine (within certain probability limits) that a person is in a REM sleep phase.

Previous workers have proposed sleep phase aware alarm clocks. Unlike conventional alarm clocks, which will wake up at a preprogrammed set time, sleep phase aware alarm clocks require users to instead set a wake-up interval—a time window during which a user wishes to be awakened. Here, the sleep phase aware alarm clock will attempt to determine if REM sleep phase occurs within this time window, either by some form of direct or indirect REM detection, or by various calculation methods.

This prior work includes Lidow, U.S. Pat. No. 4,228,806, DiLullo U.S. Pat. No. 4,832,050, Koyama, U.S. Pat. No. 5,101,831, Zaiken, Japanese patent JP3017594 (A), Hiroyuki Japanese patent JP63205592 (A), Noboru, Japanese patent JP8114684 (A), Hiroyuki Japanese patent JP1212565 (A), and Tadashi Japanese patent JP59023284 (A).

If the alarm clock determines that the user is likely in REM sleep during this interval, then the user will be awakened prior to the end of the interval, when the probability of REM sleep is high, and the user is likely to awaken comfortably. If the alarm clock determines that the user is not likely in REM sleep during this interval, then the alarm clock will instead wait until the end of the interval and then awaken the user to prevent oversleeping.

One example of such prior art sleep monitoring, regulation and planning systems is the aXbo sleep monitoring system, provided by Infactory Innovations & Trade GMBH, Austria, and discussed in Boris, EP 1139187 (A2). The aXbo system helps users fall asleep by playing soothing sounds and monitoring user movements until the cessation of user movements indicates that the user has fallen asleep. User movements are monitored by a band affixed to a limb of the user which detects movement (acceleration) and uses a radio link to transmit this movement data to the central aXbo unit which has the user interface and a computational unit, such as a microprocessor. The system continues to monitor movement throughout the night, and attempts to calculate REM sleep times, and the optimal moment for producing a stimulation signal (i.e. music, an alarm) for awakening.

One drawback of the aXbo system, and other prior art methods, is that the system's effectiveness becomes adequate only if the user's sleep lasts more than 6-6.5 hours. Part of the problem is that even if the system can predict REM sleep with absolute accuracy (100%), there is still a problem that to awaken the user at the optimal time, the user preset awakening interval needs to intersect with user's REM sleep phase. Unfortunately, as shown on FIG. 1, REM sleep is more frequent during the latter part of the night than during the first part.

FIG. 1 shows that a sleep of a typical person can be divided into cycles. Each cycle consists of one or several non-REM sleep phases and ends with a REM phase. Non-REM interval is the interval that includes an alternating sequence of sleep phases, except for the REM phase. As sleep progresses, the duration of the non-REM intervals becomes shorter and the duration of the REM intervals becomes longer. This progression occurs with each subsequent cycle during the night.

The duration of the first non-REM sleep interval (immediately after falling asleep) is not constant for all users. Rather, this parameter varies with the individual, and for certain individuals often has a defined value of approximately 70-110 minutes.

As the sleep cycles progress during the night, each subsequent non-REM interval is about 10 minutes shorter than the previous non-REM interval. At the same time, each subsequent REM interval becomes about 10 minutes longer as a rule.

FIG. 2 shows this alternation of non-REM and REM sleep intervals. Here the duration of the first non-REM interval is about 110 minutes and the duration of the first REM interval is about 10 minutes.

As previously discussed, even in such a case when the sleep monitoring system identifies the REM interval boundaries with absolute accuracy (100%), there is a probability that the awakening interval will not intersect with the REM phase interval (i.e. they will not have the same intersection times). In practice this means that the alarm clock will have to be definitely triggered at the end of the awakening interval due to the absence of the optimal awakening moment. In this case the system is not more effective than regular alarm clocks. The user continues to awaken in an uncomfortable and groggy state.

FIG. 3 demonstrates an example of the problem that occurs with sleep intervals when the awakening interval and the REM phase interval do not intersect. In this example, the duration of the first non-REM interval is 80 minutes, the duration of the first REM interval is 10 minutes, the duration of awakening interval is 30 minutes, and the awakening interval starts on the 280th minute after falling asleep.

By contrast, FIG. 4 demonstrates an example of a more ideal sleep interval situation where the awakening interval does intersect with the REM phase interval. In this example, the duration of the first non-REM interval is 80 minutes, the duration of the first REM interval is 10 minutes, the duration of awakening interval is 30 minutes, and the awakening interval starts on the 310th minute after falling asleep.

As the duration of each subsequent non-REM interval becomes shorter, and the duration of each subsequent REM interval becomes longer, the probability of awakening at the optimal moment becomes higher as sleep duration becomes longer. By contrast, with shorter sleep duration, the probability of awaking at the optimal moment is lower.

FIG. 5 shows a table that illustrates this correlation. Here, FIG. 5 shows the probability values for the awakening interval intersecting with the REM phase interval as a function of: a) sleep duration boundaries, and b) duration of the first non-REM interval.

Here the table assumes that the method for REM phase boundary detection is absolutely accurate (100%). Here the value given in the table is the probability of awakening the user at the optimal moment. Thus this represents a best-case situation. In real life, of course, REM phase boundary detection will not be absolutely accurate.

If the accuracy of the method for REM phase boundaries detection is lower, then the probability of awakening the user at the optimal moment will decrease still further. Thus the given value in the table represents the maximum probability of awakening at the optimal moment with any accuracy of the method.

In this FIG. 5 table, the awakening interval duration is taken for 30 minutes. It is also assumed that the alarm clock settings are adjusted in a way that the latest wake-up time is set within a defined sleep duration (for example from 2 to 4 hours).

The FIG. 5 table also demonstrates that the system tends to be ineffective for users that have sleep durations of less than about 6 hours. Sleeping less than 6 hours on a continual basis is a bad idea, however. Most people usually need more sleep than this, and even awakening in REM phase cannot compensate for a permanent deficit of sleep.

On the other hand, many users do need to wake up at a non-regular time on special occasions, and can get by with less amounts of sleep for short periods. Typical examples of such special occasions are travels, outdoor activities, need to communicate with people living in different time zones, and so on. In such cases, it is important for the user to stay cheerful and have a fresh and clear mind after awakening, even though the user may have gotten little sleep.

As a result, prior art systems have been hampered because, particularly due to less than optimal REM phase prediction capability, the effectiveness of these systems is not sufficient for the (relatively frequent) situation where users must sleep for periods of time less than about six hours.

### BRIEF DESCRIPTION OF THE INVENTION

The invention is an improved method and system for sleep monitoring, regulation and planning. In one embodiment, the invention may be an improved sleep phase aware actigraphy synchronized alarm clock designed for improved REM sleep phase monitoring accuracy. In a first aspect of the invention, the invention may be a sleep phase actigraphy synchronized alarm clock with an improved user interface that enables the system to be easily set up and calibrated by unskilled home users to a higher degree of accuracy (for REM phase wake-up) than prior art sleep phase alarm clocks. The system may also optionally be set up to suggest optimal times (from an optimal REM phase-wakeup) to the user to go to sleep as well.

In a second aspect of the invention, the invention may be a sleep phase actigraphy synchronized alarm clock that communicates with a remote sleep database, such as an internet server database, and compares user physiological parameters, sleep settings, and actigraphy data with a large database that may include data collected from a large number of other users with similar physiological parameters, sleep settings, and actigraphy data, and uses information and parameters obtained from this remote database to further improve the REM sleep phase prediction accuracy of the alarm clock. That is, the remote server can send sleep phase correction data to the local alarm clock that will enable the sleep phase actigraphy synchronized alarm clock to operate with greater accuracy.

In general, sleep phase correction data can be any algorithmic data (i.e. suggested algorithm coefficients, suggested equations, suggested look-up tables, suggested correction factors) that can be used to improve the accuracy of the sleep phase alarm clock's REM predictions, particularly around the wake-up interval.

Both aspects of the invention, either singly or together, will produce sleep phase alarm clocks with higher REM phase prediction accuracy. This higher REM prediction accuracy will be generally useful for all sleepers, including individuals who sleep over six hours, and will be particularly useful for individuals that must occasionally sleep for short duration periods.

### BRIEF DESCRIPTION OF THE DRAWINGS

FIG. 1 demonstrates a typical progression of human nighttime sleep phases. Here the time in hours is on the horizontal axis, and the current sleep phase is on the vertical axis.

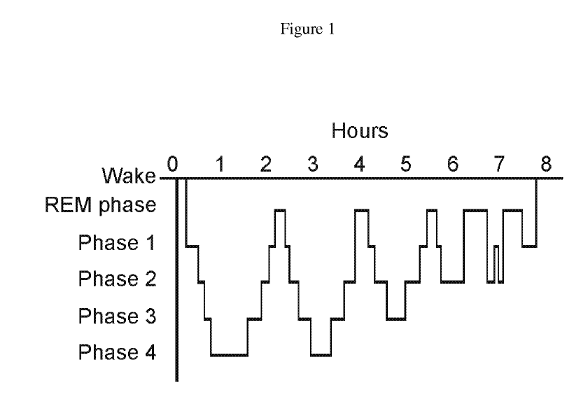

FIG. 2 demonstrates the alteration of REM and non-REM sleep intervals.

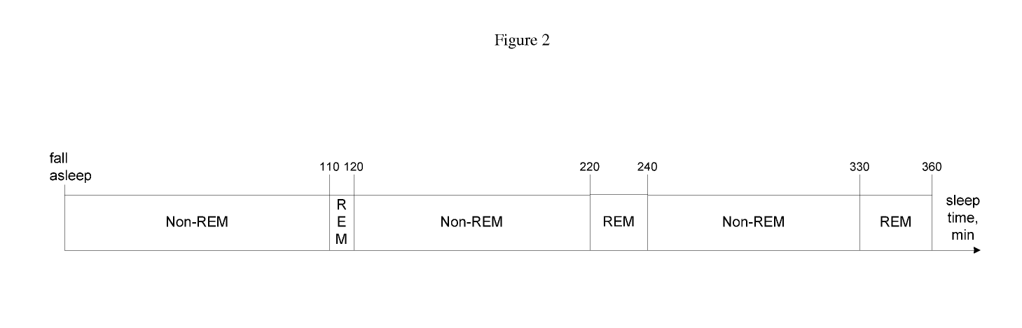

FIG. 3 provides an example of a non-optimal awakening time, in which the awakening time and the REM sleep phase interval do not intersect.

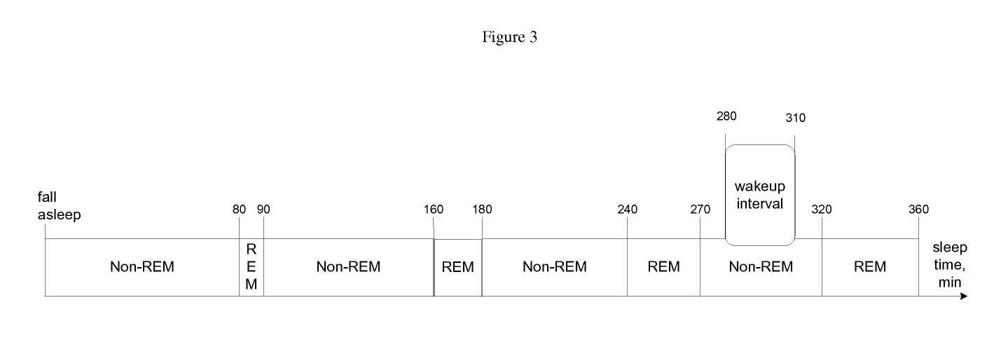

FIG. 4 provides an example of an optimal awakening time, which occurs when the awakening interval and the REM sleep phase interval intersect.

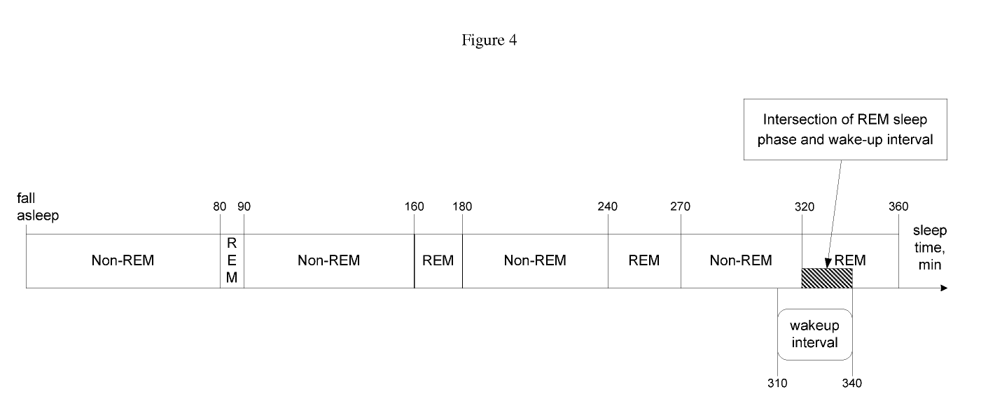

FIG. 5 shows a table of the maximum probability (when REM sleep phases are determined with 100% accuracy) of awakening at the optimal moment for a fixed sleep duration, and the defined duration of the first non-REM interval.

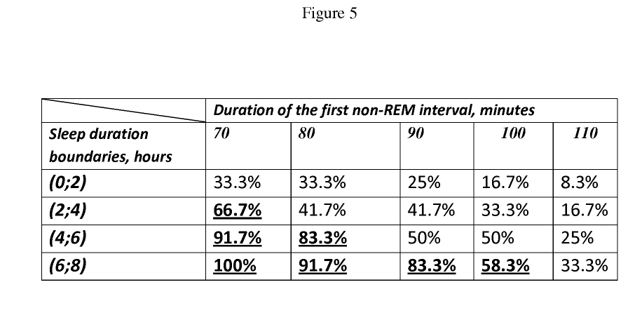

FIG. 6 shows an overview of the various components of one embodiment of the invention.

FIG. 6A shows an alternative overview of the various components of one embodiment of the invention.

FIG. 7 shows an example of a sleep calendar representation of sleep data.

FIG. 8 shows a column graphical representation of sleep data.

FIG. 9 shows a circular graph representation of sleep data.

FIG. 10 shows a flow chart of significance of individual sleep phase characteristics on the sleep phase alarm clock software algorithm.

FIG. 11 shows an example of user analysis when the duration of the REM phase is known with lower accuracy (usually due to the absence of much historical data on the user's REM sleep patterns).

FIG. 12 shows an example of user analysis when the duration of the REM phase is known with higher accuracy (usually because of more historical data on the user's REM sleep patterns is available).

FIG. 13A shows a flow chart showing how the device's software may handle these general objective factors and objective daily factors. This also shows the dependencies between factors impacting sleep and sleep characteristics.

FIG. 13B shows a flow chart of how the system may utilize global individual factors, changes in global individual factors, and daily objective factors in sleep analysis calculations.

FIG. 13C shows a flow chart of how the remote server can obtain, process, and transmit sleep data to and from various local devices for awakening.

FIG. 14 shows a graph showing the interdependence and data redundancy between several types of data collected from group 1 (fully reporting) system users.

FIG. 15 gives an example of the type of analysis that is possible when there is a bidirectional correlation present between the “Detailed daily movements data” on one side, and “Daily factors data” and “Global factors changes” on other side.

FIG. 16 shows how the remote server system may analyze group 2 (partially compliant) users.

### DETAILED DESCRIPTION OF THE INVENTION

The system will generally be comprised of multiple components. These components will include 1) one or more actigraphy (movement sensors), typically limb movement sensors, 2) a central alarm clock device (device for awakening) which will usually be comprised of a visual display, at least one microprocessor, user input devices (i.e. a touch sensitive display, buttons, or user input mechanism), a device, such as a short-range radio receiver or transceiver to receive user limb motion data from the movement sensors, memory to store programs and data to run the display and perform sleep phase calculations, a speaker or other sound generation device to play soothing sounds when the user is going to sleep, and/or generate sounds to wake the user up. The clock device will also often have network interface devices, such as an Ethernet connection, telephone connection, or other connection to allow the device to send user parameters and actigraphy data (or other user REM data) to remote servers, and to obtain REM sleep phase correction data from remote servers. The system will also generally have 3) a remote sleep data server, often handling multiple users, which can act as a central storehouse for physiological parameters, actigraphy data, and sleep schedule data from a large number of users. Often this remote sleep server will compare an individual user's data with a database comprised of individuals with similar parameters, and based upon this comparison, as well as a record of the results of previous data obtained from the user, send sleep phase correction parameters to the local alarm clock device. The remote sleep server can also store other data as well including external parameters, such as time of year, user environment, weather, and where past experience shows that this external data can be useful, use this external data to adjust the sleep phase correction parameters on the local alarm clock device as well.

The device for awakening can also contain other fixtures, such as indicators for indicating power on/off status, displays showing the remaining battery life of the motion sensor (or device for awakening itself if this device is battery powered), on/off switches, etc.

As previously discussed, in order to obtain as much user REM data as possible in a relatively unobtrusive manner, the system will usually have at least one user actigraphy (movement) sensor to measure user movements during sleep. Usually these actigraphy sensor(s) will be limb movement sensors such as an arm or leg band equipped with an accelerometer or other movement detecting sensor, often a battery, and a device, such as a short-range radio transmitter or transceiver, capable of transmitting the user movements to a nearby receiver or transceiver. This receiver or transceiver will often, but not always, be located on or near the main body of a local, microprocessor equipped, sleep phase alarm clock device.

To conserve the battery lifetime of the movement sensor, data compression and buffering can be used when transferring data between the movement sensor and the device for awakening (sleep phase alarm clock). The movement sensor will often use industry standard low power radio transmission technology, such as Bluetooth®, Zigbee, Wi-Fi, or even one of the various RFID protocols.

In order to get the highest REM phase prediction capability as possible, in some embodiments of the invention, the invention will also assist the user in falling asleep by playing soothing sounds or noises, such as music, friendly conversation, white noise, nature noises, and the like. The device may optionally assist the user in selecting an optimal moment for going to bed by suggesting times on a visual display, or spontaneously playing “suitable bedtime noises” based upon the status of the device's internal REM phase prediction sensors. Once the device has detected that the user has fallen asleep, for example by detecting a reduction of limb movements, it will usually be programmed to then stop playing the “bedtime noise” sounds.

Thus in one embodiment, the system will provide a method for sleep monitoring, regulation and planning that comprises assistance falling asleep by playing soothing sounds by the device for awakening (alarm clock device) until the moment the user falls asleep. The alarm clock device may detect the optimal moment for awakening the user by monitoring data from the motion detector and using this data to determine an optimal time for producing an alarm, light, music, vibration or other stimulation signal. In fact, the device for awakening can have a general purpose plug that can supply power or turn on any suitable attention getting device, including heaters, coolers, fans, etc. As another alternative, vibrating motors or other vibration device can be used to awaken sleepers without disturbing other nearby persons.

The device for awakening (alarm clock device) will also have calculation means (such as a microprocessor) and memory means (i.e. random access memory, flash memory, or other type of memory) to store and process user limb motion data, usually obtained by a short-range radio link) from one or more limb motion monitoring actigraphy sensor(s). The alarm clock device will be capable of processing user input data as to sleep schedules and motion data independently. However, in an improvement over prior art sleep phase alarm clock technology, the alarm clock device of the invention will additionally be able to connect up with a remote server (global system server) containing a vast amount of sleep data and other physiological data collected from a large number of users, send data to this server, and in turn use data transmitted from this global server to improve the accuracy of the alarm clock devices REM sleep phase predictions, resulting in improved user satisfaction. Other data, such as amount of user physical activity, mental workload, stress, alcohol or stimulants, medication, time zone change (due to travel), sickness, and other sleep affecting conditions may also be entered into the remote server, and used to further refine the sleep phase calculations.

At the same time, because the alarm clock device has its own local REM sleep phase prediction capability, the system can fail in a graceful manner in the event that the connection with the remote (global) system server is lost. In the event of a loss of data connection with the remote system server, the local alarm clock device will continue to function, of course without the benefit of the increased accuracy from the remote system server. In the event of an intermittent loss of data connection, the device will generally function at an intermediate level of accuracy.

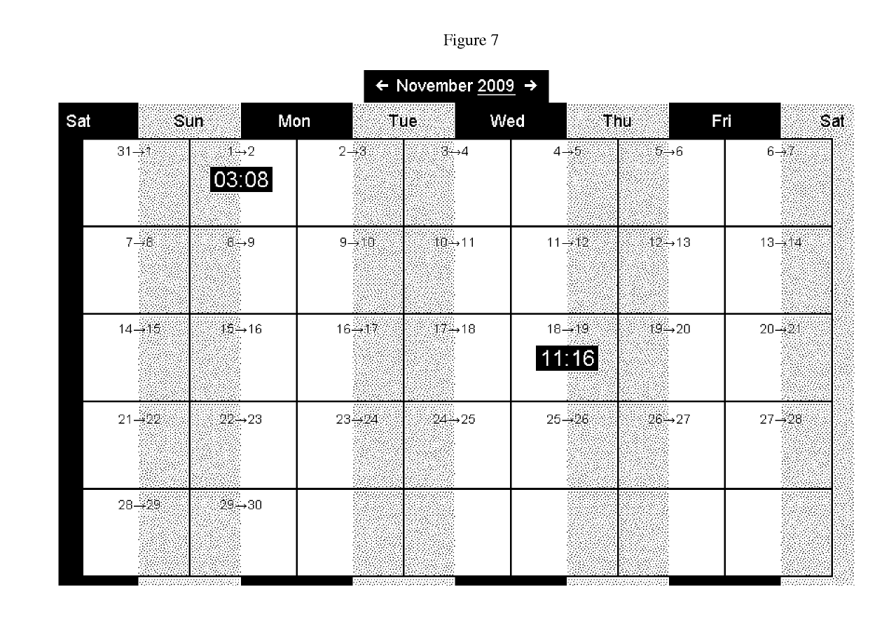

Using either a display on the local alarm clock device, or alternatively a computer connection to the remote database, the user can view his or her history of sleep data anytime, as well as receive information on sleep duration and quality, and movements during sleep. The user may evaluate their wellbeing basing on this data, and annotate (add to the database data) with a subjective evaluation of their own wellbeing. Thus, for example, if the alarm clock made a particularly good wake time suggestion, the user can annotate the data from this day with a positive comment by clicking a “felt great” button or other input category. Conversely, if the alarm clock functioned less well, and the user woke up feeling bad, the user could annotate the data from that day by clicking an appropriate “feel bad” button or other input category. Additionally, alternatively, or optionally, the system may calculate a sleep quality index or score, and present this to the user as a default option, in which case the user needs only to enter input into the system if the default sleep quality index or score is incorrect. Basing on this data, the system can then recommend the user one or several variants of suggested time for going to bed, so that the awakening time matches REM sleep phase. Examples of some of these potential data displays are shown in FIGS. 7 to 9.

To facilitate data entry, in some embodiments of the invention, it will be useful to make the display a bit mapped video display, such as a bit-mapped liquid crystal display, bit mapped electronic paper, and the like. Often, it will be useful to use a touch sensitive video display as well, so that the user may enter data directly onto the display by touching appropriate locations.

A specific application of the method for sleep monitoring, regulation and planning is shown below. Here the alarm clock device is termed a “device for awakening”, and the limb mounted actigraphy sensor is termed a “motion detector”.
1) In the evening, before going to sleep, the user powers on the motion detector 3 and the device for awakening 1 (if they were powered off) and ensures that a radio channel connection is established between them with the help of indicators on the motion detector 3 and/or the device for awakening 1.
2) The user attaches the motion detector 3 to a limb.
3) The user sets a desired time for awakening and a desired awakening interval with the interface of the device for awakening 1, and this information is saved in the memory of the device for awakening 1 and sent to the motion detector 3.
4) Based on previously discovered individual characteristics of the user's sleep, the device for awakening 1 calculates one or several variants of the optimal time for going to bed so that the planned awakening moment would intersect with REM sleep phase with maximal probability.
5) The device for awakening may optionally also calculate variants of the optimal time for going to bed, and suggest these times to the user. The device for awakening can also display or otherwise indicate whether the current time is an optimal time for going to bed.
6) After considering the suggested variants, the user goes to bed at hopefully the closest optimal moment for going to bed, and sets the “I am going to sleep now” mode on the device for awakening 1.
7) After setting the “sleep” mode the device for awakening 1 may optionally play a soothing melody or other pleasant noise to help the user to fall asleep.
8) Depending upon user preferences playing soothing melody can often increase the probability that the user will fall asleep during a certain defined time after going to bed, for example 10-20 minutes. Here, falling asleep can be detected with some probability using input data from the motion sensor, since typically users will move less after initially falling asleep. Each individual may have his or her own average time for falling asleep, and here the device can accumulate data and gradually track this time with higher accuracy as user data accumulates. Although users may elect to set the device to allow them to go to sleep without any noise or music, if this option is selected, the possibility that the user will not be able to fall asleep during certain time increases. Thus the system may function with less accuracy in this situation, but the system can be set to respect user preferences here.
9) After switching to the “sleep” mode the device for awakening 1 sends the corresponding command to the motion detector 3, and the motion detector 3 also switches to the “going to sleep now” (sleep) mode.
10) While in the “sleep” mode, the motion detector tracks direction and acceleration of a motion of a limb of the user by built-in accelerometer 4. Received data is processed with the help of the processor 5 by the embedded software 2. Analyzing the processed data, the processor 5 permanently monitors user's sleep and detects moments when the user was completely awakened during the night or in the morning, probable transitions into and from REM sleep phase, and as a result the optimal moment for awakening within the defined interval is identified.
11) Processed data on user's sleep is sent to the device for awakening 1 with the help of the radio modules 6, 11 of the motion detector 3 and the device for awakening 1.
12) If the motion detector 3 identified the optimal moment for awakening the user, it sends the command “wake up” to the device for awakening 1. Thus for example, an ideal time for awakening is when the motion sensor directly indicates that the user is in REM sleep. Often, or course, this motion data will be inadequate to make an exact determination, and thus the device will function in these cases by making interpolations and extrapolations from previous data. In the event that an absolute “wake up” time (end of the wake-up interval) is reached, the device will also wake up the user, regardless of sleep phase.
13) After receiving the command “wake up”, the device for awakening 1 produces stimulating signal to wake up the user. As previously discussed, this can be an alarm sound, music, turning on a clock radio or television, and can also be an alternative stimulating signal such as one or more lights.
14) The device for waking up (alarm clock) will also establish a connection with a remote server, often through the internet or other networking system. Data on user's sleep transmitted to the server 7 and is stored in the database 8. With the help of the software 10, a resource-intensive analysis of the user's sleep data is performed centrally on the server. In many situations, the server will have access to much more data than the alarm clock device could access. Thus as a result, the server can perform more complex analysis, which would be hard, unpractical, or impossible to perform otherwise. As one example, during this centralized analysis of the user sleep data on the server 7, other users' sleep data can also be considered, which increases the efficiency of the analysis. As one example, the central server can quickly match the users with other users with similar physiological or other parameters, and identify an appropriate “sleep group” to classify the user. This allows cutting time of identifying individual characteristics of sleep for each user compared to the example when the analysis is only performed with the help of the local device for awakening 1 containing sleep data of only one or several users.

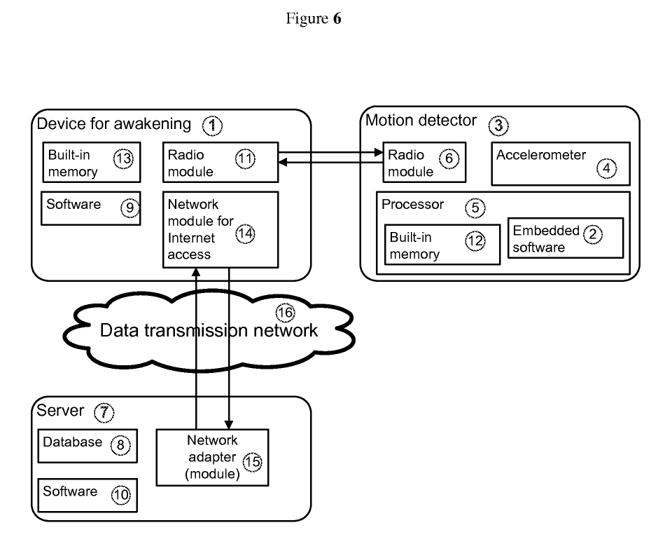

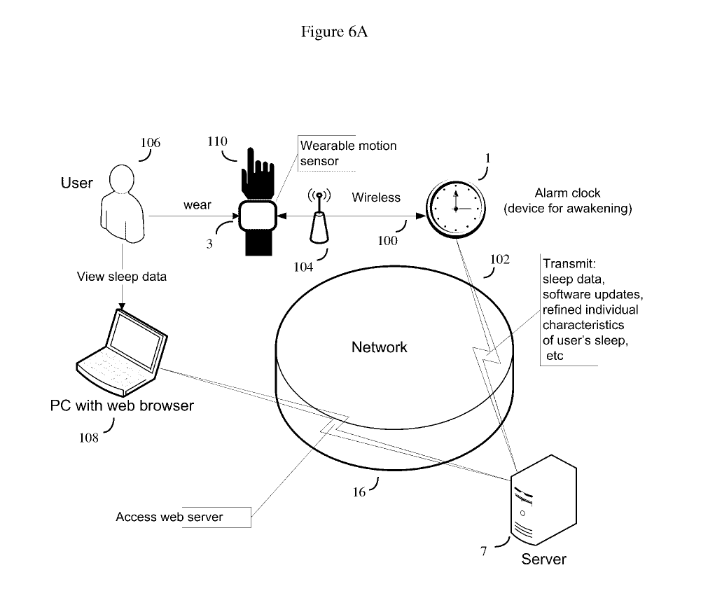

FIG. 6 shows an overview of some of the major components of one embodiment of the invention. Here (1) is a device for awakening (sleep phase alarm clock), (2)—embedded software, (3)—a motion detector, (4)—an accelerometer, (5)—a processor, (6)—a radio module of the motion detector, (7)—a server, (8)—a database of users sleep, (9)—software of the device for awakening, (10)—software executed on the server, (11)—a radio module of the device for awakening, (12)—a built-in memory of the motion detector, (13)—a built-in memory of the device for awakening, (14)—a network module of the device for awakening, (15)—a server network module, (16)—a data transmission network.

FIG. 6A shows an alternative overview of some of the same major components of one embodiment of the invention, previously shown in FIG. 6. In contrast to FIG. 6, which showed some of the interior portions, components and data flows of the invention, FIG. 6A focuses more on what these components look like from the outside. In FIG. 6A, the device for awakening (1) is shown as an alarm clock, and indeed in some embodiments of the invention, it may be useful to have the default image shown on the device's screen in fact resemble an analog or digital clock face. Because FIG. 6A focuses on the outside, the software for the device for awakening (9), the built-in memory (13), and the radio module (11) is not shown, however they are shown functioning via the radio link (100) and data transmission network data link (102) lines. Here the radio modules (11) and (6) are simply drawn as a single module (104), although in reality, the device for awakening or sleep phase alarm clock (1) will usually have an internal radio module (11), and the motion detector (3) will also have its own internal radio module (6).

As discussed elsewhere, the user (106) will be able to transmit and view various types of sleep data, such as global individual factors and daily objective factors to and from the remote server (7) either by way of a user interface on the device for awakening (1) or by alternate means, such as a network connected personal computer (PC) and web browser (108). The wearable motion sensor (3) will usually be connected to an arm or leg (limb) (110) of the user (106).

FIG. 7 shows a table that provides graphic expression of user's sleep data, here represented as a sleep calendar. Each cell represents a night with user's sleep data, such as:
Date (two dates of the month are given—the date when the user woke up and the previous one, for example the night of 3^{rd}-4^{th} November is denoted as “3->4”);
Number of hours the user was sleeping;
Quality of sleep or feeling after awakening is marked with corresponding color;
Days of the week specified in a heading line above the cells are located between the nights helping the user to easily read the calendar.

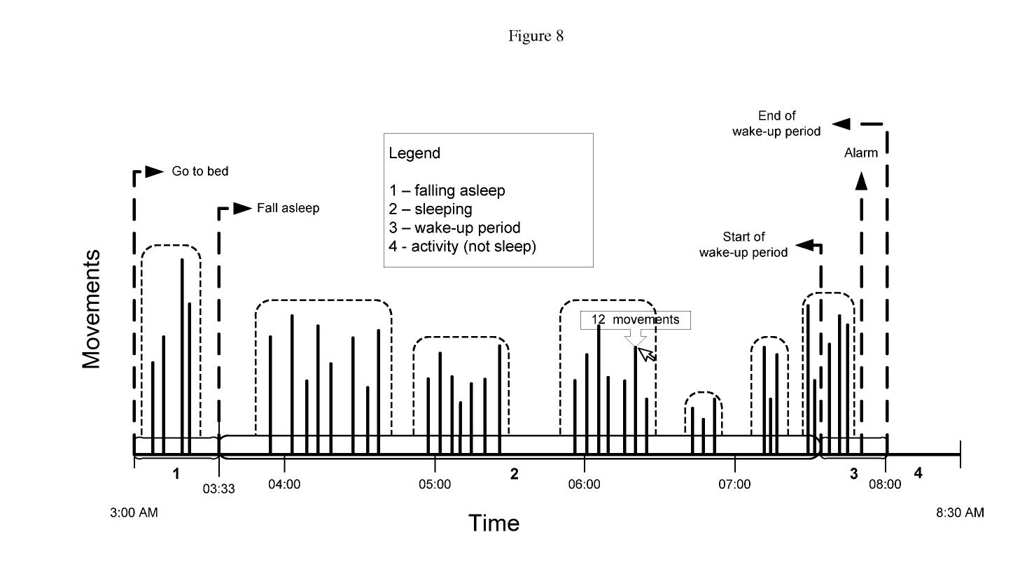

FIG. 8 shows one of possible graphic representation of user's sleep data. This is shown as the sleep column diagram area, which can be represented by a display on the sleep phase alarm clock, or alternatively on a web browser of a computer or other device connecting with a global sleep database. Here the time is represented on the X-axis, and the amount of movements per minute of a person sleeping is reflected on the Y-axis. For better visual perception, several subsequent movements can be joined into a single background color column. Additionally, for better visual expression, the Y-axis can have a logarithmic scale, rather than a liner scale.

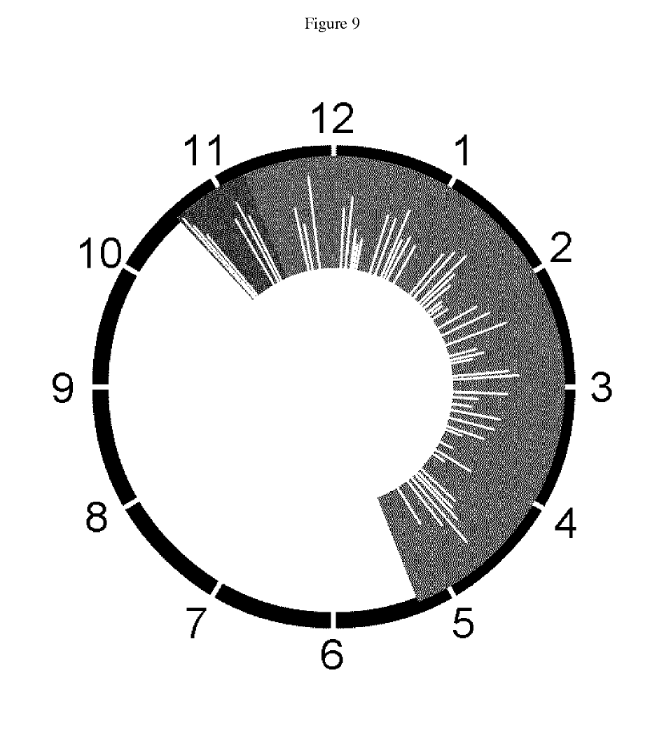

FIG. 9 shows variant graphic representation of user's sleep data—in which the device shows the sleep data on a circular sleep graph.

The system will normally be designed to be robust to various operating errors. For example, when there is no movement from the user's motion detector—either due to lack of motion, or due to lack of signal, the device will be set to wake up the user at the end of the preset awakening interval.

### Robustness Against Connection Interruptions:

In several everyday situations, connections between the device for awakening (often mounted on a bedside table near the user) and the user's motion detector (usually placed on the limb of a user by a band) can be interrupted due to a discharged battery, movement of the user to a different room, or because the user accidentally or deliberately detaches the motion sensor.

In such cases system can be programmed to ignore the bad input data, and awaken the user at the end of the preset awakening interval.

Similarly, as previously discussed, although the device for awakening will be designed to frequently synchronize and exchange data with a remote global server in order to obtain refined individual characteristics of user's sleep and software updates, this connection also can be designed to be robust. In the event of connection failures, the software in the device for awakening can be designed to simply use either default sleep parameter data, the last set of user sleep parameter data uploaded from the server, a time average of typical user sleep parameter data, or other fallback dataset.

Various embodiments of the system are possible. For example, in one embodiment, the remote server can supply various graphical interfaces, such as the interfaces in FIGS. 7-9, to users by various means including a web server/web browser mechanism. This interface can provide users with a comprehensive overview of a sleep calendar, as well as more detailed sleep information in a column format or circular graph format. These can provide information such as (1) the interval of falling asleep; (2) the interval of sleeping; (3) the interval for awakening set by the user; and (4) the intervals of activity (not sleep)—which is outside the other intervals on the drawing, on the right and on the left.

The time of the following events such as the time when the user went to bed, the time when the user fell asleep, the beginning of awakening interval, the moment of awakening—when the alarm clock was triggered, and the end of awakening interval can also be provided.

The circular graph can also represent aggregated values of above mentioned moments and intervals, collected during certain periods of time, for example:
the interval defining the boundaries of awakening the user during the last week or month;
the average time of awakening the user during certain time;
minimum/maximum and average time when the user falls asleep, basing on the data for certain period;
maximum deviations from the typical sleep schedule for certain period.
   Smooth variations of aggregated values within the graph can also be marked on the circular graph with smooth color shift or other graphic effects.

In many situations, it will be useful to be able to configure the device for awakening to obtain software updates, either from the same remote server that holds the sleep database, or from some other source. Examples of useful functions that can be added by software updates include system functionality extensions such as “nap” modes, support for more external devices to provide stimulating signals to awaken the user, new melodies or other sounds for falling asleep, and so on.

Since one of the unique aspects of this invention is the remote server, this aspect will be discussed in more detail.

### Server-Based Analysis and Refinement of Individual Characteristics of Many Users:

In general, in order to set up a remote server capable of performing more refined and accurate REM sleep phase analysis and predictions, and that which can send sleep phase correction data to a local device for awakening in order to make the local device (sleep phase alarm clock) operate with greater accuracy, a number of considerations must be addressed. These include:

1: Identification of a user's sleep characteristics by analyzing individual data on the user's movements during sleep.
2: Analysis of the impact of objective factors on sleep characteristics.
3: Extended analysis of sleep characteristics using extended information available from many users.
4: Analysis of the effect of missing objective factors on sleep prediction
Since the goal is to increase the accuracy of the detection of REM phase boundaries, individual sleep characteristics are critical. This requires both detection of user REM phase boundaries, as well as detection of the times when the user wakes up at night.

In general, the accuracy of detection of REM sleep boundaries depends on user's sleep characteristics, such as:
1. Duration of the first non-REM and REM sleep intervals
2. Dynamics of sleep cycles duration (dynamics of decrease of non-REM interval duration and increase of REM interval duration along the night)
3. User's movements intensity during various sleep phases
4. Threshold value of acceleration, which allows exclusion of micro movements, caused by breathing, heartbeat, meter accuracy, etc.
5. Probability of complete and incomplete awakening of the user during the night. In case of complete awakening the sleep cycle starts from the beginning, and in case of incomplete awakening—continues. It can also be defined with the help of movement analysis.
6. Typical duration of falling asleep.

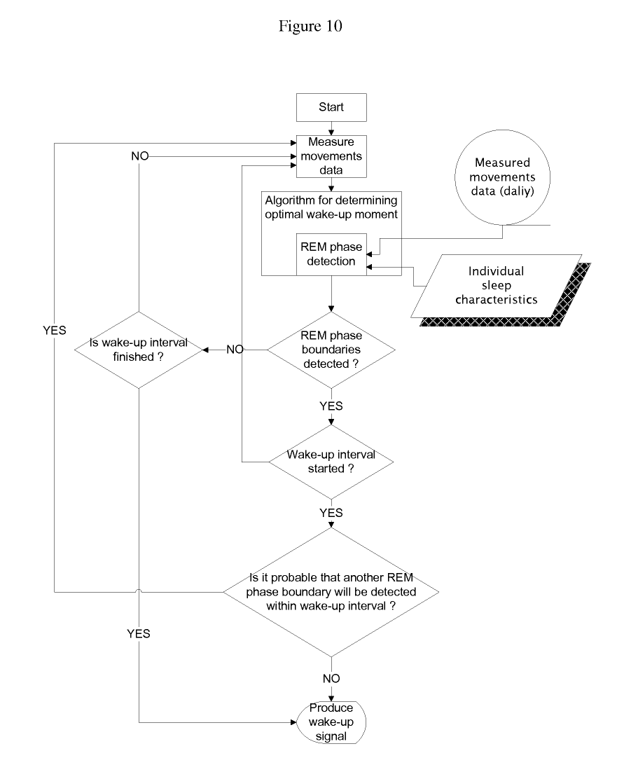

The invention will take these characteristics into account in an algorithm that determines the optimal wake-up time parameters. A flow chart of this diagram is shown in FIG. 10.

Individual characteristics of user's sleep are partially predetermined by individual physiological and psychological characteristics of the user, the user's environment and other events. In general, the user's individual sleep characteristics can be identified by sequential analysis of the user's movements. Here more data is better, because when data on a user's sleep patterns are accumulated over many days, the system can more accurately predict REM sleep patterns and thus more accurately determine optimal times to wake up the user.

### Example 1

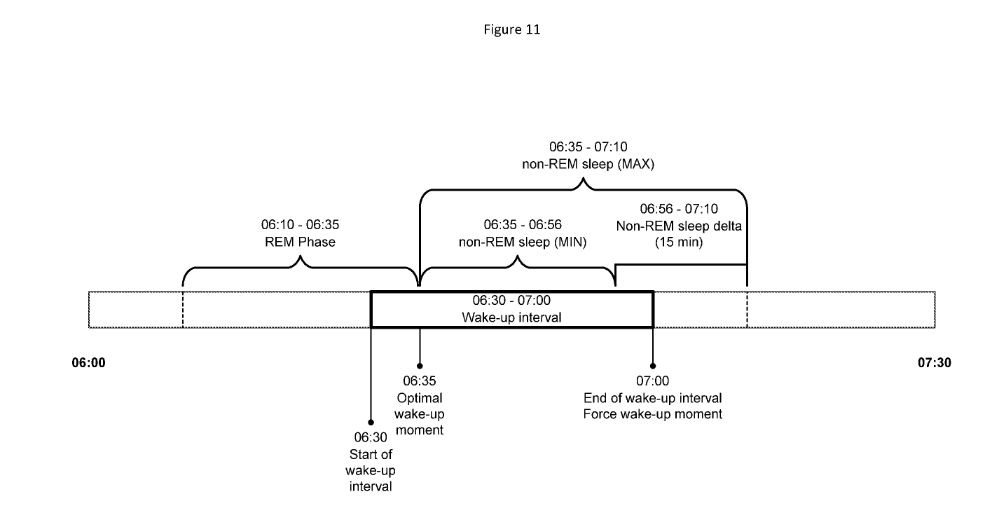

The user has only used the system for several days, and the system does not yet have enough accurate data on the user's REM phase and non-REM interval duration at the end of sleep. In this example, if the user has set a wake-up interval to the 6:30-7:00 AM interval, and the system has determined that the exit from REM phase occurs at 6:35 AM, then it is obviously better to wake up the user at this moment. This is because there is a probability that the subsequent non-REM interval will be longer that 25 minutes, and the system will be forced to wake up the user at 7:00, which may be a at non-optimal wake-up time. This situation is illustrated in FIG. 11. Here, due to limited data, the duration of the non-REM interval is known to within about 15 minutes accuracy, and the system will conservatively determine that the optimal wake-up moment is 6:35 AM.

### Example 2

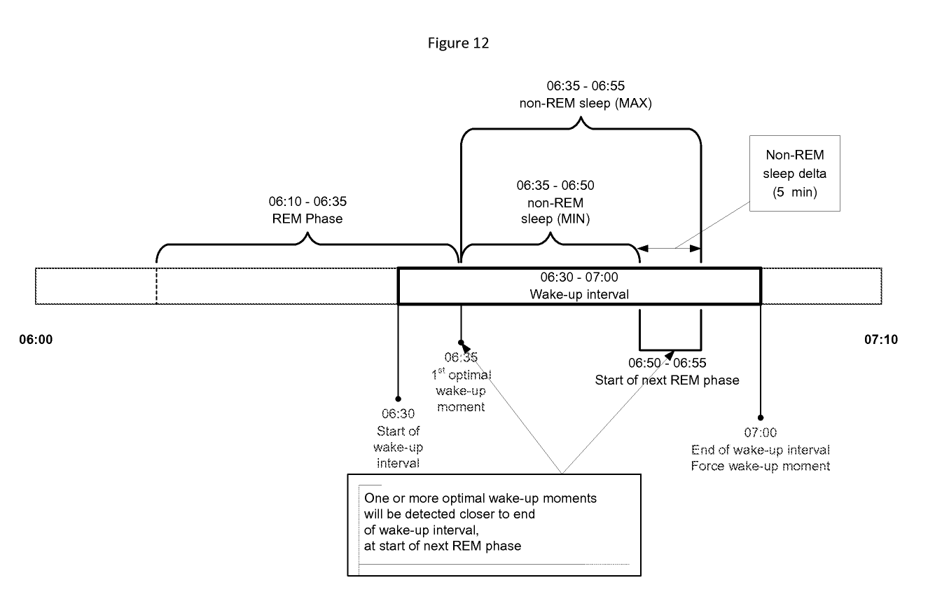

In this example, the user has used the system for a longer period of time, and the system now has information that the duration of the non-REM interval (i.e. spacing between REM phases) is 15-20 minutes at the end of sleep. The user has again set the wake-up interval 6:30-7:00 AM, and the system has again determined the exit from REM phase will occur at 6:35 AM. Because the system now has more information, the system also knows that the user will enter the next REM phase at 6:55 AM, which is still within the target, wake-up interval. Because the system now has more information, the system can give the user more sleep while still accomplishing the wake during REM phase objective. Thus the system will not wake the user up at the first moment of exiting from REM phase at 6:35 AM, but will instead wait until the moment of entering the next REM phase (closer to 6:55 AM). This will allow the user to benefit from an additional 15-20 minutes of sleep, in contrast to the first example. This is shown in FIG. 12.

In these examples the analysis algorithms are not particularly complex, and they do not require either considerable computational resource or other data on other users sleep patterns. Thus these algorithms can run on the local device for awakening even when access to the remote server is unavailable. Thus these are good examples of default “no extra data” algorithms that can initially run on the device prior to hooking up to a remote server, and/or when a server is unavailable.

### The Impact of Objective Factors on Sleep Characteristics:

Other objective factors can also influence sleep. Here we can obtain information on the presence of such factors by interviewing the user and obtaining data on these factors. To do this, the system should ideally have a good user interface.

One type of general user information (objective general factors) is usually obtained when the user starts using the system, and does not need to be frequently updated unless there is a significant change in any of these parameters. Examples of objective general factors (the ones that change rarely) impacting sleep are:
1. Anthropological data (height, weight, gender, age)
2. Lifestyle and schedule (fitness, sports, work, type of work, nutrition, etc.)
3. Sleep environment (temperature, humidity, bed quality, presence of other people in bed, room or house)
4. Geographical location (climate, solar day)
5. General physical state (health)

In addition to these objective general factors that do not change very often, there are other factors that can vary on a daily basis that also impact sleep. Examples of these objective daily factors include:
1. User sports activity,
2. User stress levels,
3. User consumption of pharmaceutically active substances such as alcohol, nicotine, narcotic drugs, and medications
4. User medical treatment
5. User physical or mental overstrain,
6. User food consumption levels, such as a heavy meal before sleep,
7. Abnormal user sleep schedules, such as sleeping during the day,
8. Exhaustion

Since this data is again user specific, it can still be handled by either the local device for awakening, or the remote server. Often the local system will obtain and store information about the user's most recent sleep quantity, as well as data on the user's sleep quality for a recent period (for example the past several days).

In the morning after awakening the user provides evaluation of how he feels and the sleep quality, also by means of feedback communication. Since, due to human nature, some users may tend to give input only when the device has made some improper sleep phase calculations, while others may want to give input only when the system works well, the system may be set up with various default mechanisms to allow the user to set that in the absence of input, the results are good, or the results are bad, or the results should be given no weight.

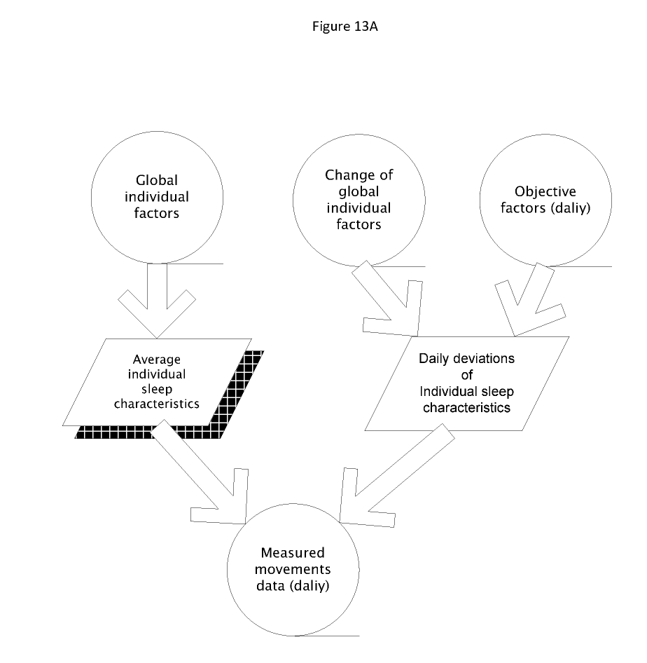

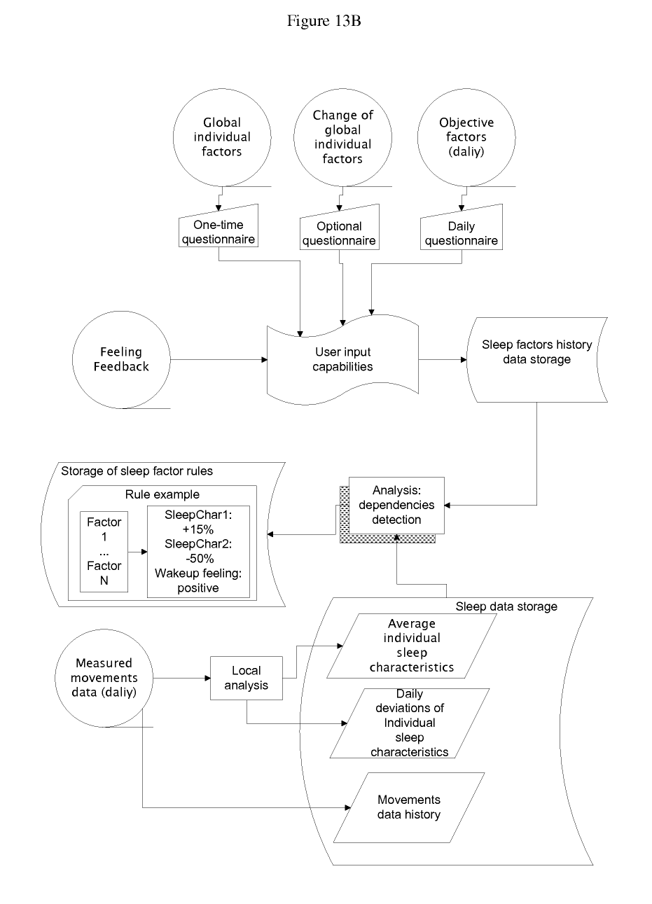

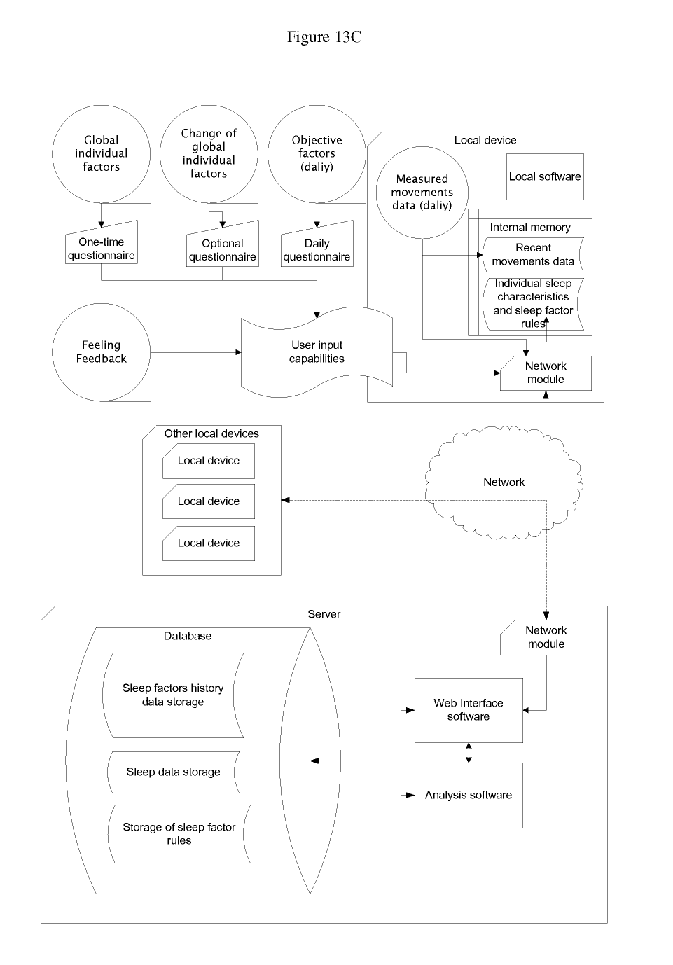

FIG. 13A shows a flow chart showing how the device's software may handle these general objective factors and objective daily factors. This also shows the dependencies between factors impacting sleep and sleep characteristics.

In some embodiments of the invention, the invention can provide one or more user interfaces to allow users to input this additional data. By analyzing this data, dependencies between this data and factors impacting sleep can be determined, allowing the system to perform with still higher accuracy.

Examples of these dependencies are shown in the tables below. Here the particular objective factor, the degree of impact of a particular objective factor (or combination of factors) on sleep characteristics 1-5 and user's feeling and sleep quality are noted.

Tables 1-2: examples of typical daily conditions, frequency of occurrence (Table 1) and their typical impact on user sleep conditions.

TABLE 1
typical daily conditions and rough frequency of occurrence

| | Frequency of |
|---|---|
| Condition | occurrence |
| Sleep quantity and quality | Average |
| Stress | Not present |
| Sports | Seldom |
| Mental overstrain | Present |
| Physical overstrain | Not present |
| Heavy meals | Not present |
| Day sleep | Not present |
| Exhaustion | Present |

TABLE 2
impact of various daily objective sleep factors
on user sleep quality and sleep characteristics

| Combination | | | Impact on |
|---|---|---|---|
| of factors | | | sleep quality |
| (deviation | | | and on how |
| from typical | Impact on sleep | Degree of | the user |
| conditions) | characteristics | deviation | feels |
| Sports | Increase of non-REM | by 15% | Positive |
| Physical | phase duration | | |
| overstrain | Decrease of falling | by 50% | |
| | asleep interval | | |
| | Decrease of wake-up | by 70% | |
| | times during the night | | |
| Exhaustion | Increase of falling | by 30% | Negative |
| | asleep interval | | |
| | (insomnia) | | |
| | Decrease of wake-up | by 35% | |
| | times during the night | | |
| Stress | Increase of falling | by 5 times | Negative |
| | asleep interval | | |
| | (insomnia) | | |
| | Increase of wake-up | by 3 times | |
| | times during the night | | |
| Heavy meals | Increase of falling | by 3 times | Negative |
| before sleep | asleep interval | | |
| | (insomnia) | | |
| | Increase of wake-up | by 3 times | |
| | times during the night | | |
| Exhaustion | Increase of non-REM | by 5% | Neutral |
| Lack of | phase duration | | |
| sleep for | Decrease of falling | by 50% | |
| previous days | asleep interval | | |

The above schemes are still simple enough that they can be either performed on the relatively small amount of computational capability in the local device for awakening—that is, these could, for example, be run on the local device's microprocessor(s). Alternatively these schemes may also be delegated to a remote server when it is available, and when the remote server may have additional refinements to the calculation schemes to improve accuracy.

FIG. 13B is a scheme of the analysis of the various factors impacting sleep. The figure shows a flow chart of how the system makes use of the global individual factors, changes in the global individual factors, and the daily objective factors to perform its sleep analysis calculations.

However as the computational schemes and algorithms become still more complex, and particularly as the computational schemes and algorithms require access to additional data, such as a complete database of user's sleep data and patterns, then increasingly it makes sense to delegate more complex algorithms to the remote server.

Various algorithms can be used to take objective factor input data and determine particular dependencies and parameters most useful for producing higher accuracy sleep phase prediction algorithms. One useful method is to one or more “black box” analysis methods such as, for example, supervised learning methods. These methods can include back-propagation artificial neural network algorithms, and association rule learning algorithms.

For example, consider a “black box” analysis (or supervised learning method) that operates by the back-propagation neural network method. These methods work even when the exact model for combining input factors is unknown. Here, numeric data is provided for the algorithm as pairs: (input data, the output), where an input data can be a rule values vector, and an output data is the scalar value. In the case when the output data is a vector, the algorithm is applied several times, separately for each scalar element of the output vector.

This type of algorithm correlates input data and the output for each given pair, and tries to find complex dependencies between input data and the output. That is, the method practically tries to reproduce the model without assumptions about its essence. It is clear that when ambiguous values are put in, the method quality will be low. Other known algorithms can be applied to check quality of the input data.

In our case the input data includes objective factors impacting sleep, and the output includes change of user's sleep characteristics and user's feeling.

This type of algorithm is essentially a more general type of mathematic interpolation method. It is quite useful for revealing hidden (unobvious) dependencies.

For example, if a factor such as “heavy food” occurs before sleep, as a rule it has a negative impact on sleep quality. But if this factor is combined with a different factor, such as outdoor activities, the general impact of these factors combination might end up being positive.

After the pair analysis (input data; the output) is completed, the system can be used to make predictions that would otherwise be difficult or impossible to do.

For example, when the system has information on a user's sleep duration for the previous days, and also has various user indicated factors that can impact sleep, then based on the input information, the system can predict the extent of the probable deviation of the user's sleep characteristic from normal. This information in turn can be used to increase the accuracy of detection of the REM sleep phase boundaries. In turn, the device for awakening can use this better prediction to make a higher quality determination of the optimal wake-up moment depends. Users will be able to sleep longer, on the average, yet still not wake up feeling bad.

Here again, there is some degree of flexibility as to where this algorithm can be run. It could be run on the local device for awakening (possibly as a simplified version), however because this is computationally intensive, and because it depends upon accurate correlation data, this algorithm may in many embodiments be preferably run on a remote server, and the results uploaded to the local device for awakening.

The computational trade-offs for this type of algorithm are shown below in Table 3.

TABLE 3
computational trade-offs for “black box”
analysis (supervised learning algorithms).

| Characteristic | Value |
|---|---|
| Resource-intensiveness of | High |
| the algorithm | |
| Algorithm complexity | Relatively high |
| Input data type and means | History data on user's movements during sleep |
| for its acquisition and storage | History data on sleep characteristics and their change |
| | Data on factors impacting sleep |
| | Feedback communication: Data on wake-up feeling |
| Advantages and | Advantages: Capability to take objective factors into |
| disadvantages in comparison | account and reveal their impact on sleep |
| with other algorithms | Disadvantages: Relatively complete information on |
| | factors is required, i.e. regular feedback |
| | communication with the user. |
| | User interface means are required |
| Summary on the efficient | As a rule embodiment of supervised learning methods |
| method for algorithm | requires considerable computational resources. |
| embodiment. | Computational complexity of a method depends on |
| | data dimension and quantity. The method also |
| | requires availability of whole history data on user's |
| | sleep, factors and evaluation of feeling. |
| | Computational resources and memory capacity (both |
| | permanent and operative) of local device might be |
| | insufficient for performing such analysis, thus it may |
| | be better to use an external server or local |
| | workstation (PC). |

Analysis Using Data Obtained from Many Users:

In general, with adaptive learning methods, the more information that is available, the better. Thus in general, it is highly advantageous to perform such adaptive learning algorithms (e.g. FIG. 13C) on a remote server, because there data from a large number of individuals can be aggregated, similar users' characteristics can be found, and this data can in turn be used as a benchmark (i.e. starting point, or initial point) for calibration (refinement) of sleep characteristics for new users.

### Example 3

Here multiple users in multiple locations use their various local devices for awakening, as well as using the user interface in their local device for awakening (or alternatively an alternative means such as a web browser) to enter in their various general objective factors and objective daily factors into the remote server. The various local devices for awakening also transmit additional information, such as the record of user movement during the night obtained from the various movement sensors, which can be used to determine REM sleep stages. Additional information transmitted can include some or all of the various user settings for the local device for awakening—i.e. wake-up time windows, snooze settings (if any), and so on.

Here the database on the remote server will obtain a relatively large amount of data. When a new user, (preferably the one who at least provides information on global individual factors), joins the system, the search for optimal values of this user's individual sleep characteristics will not have to start “from scratch”, but rather from certain initial values taken from already existing user database. For example, if the user states that he is a 35-year-old man, height—175 cm, weight—80 kg, married, with a sedentary job, non-drinker, not practicing regular sports, and has no chronic diseases, the system will find the most similar set of users. Using this data obtained from this similar set of users, more accurate individual sleep characteristics are already known, and the system will use these values as initial values. The remote server can then upload these values to the user's local device for awakening, and the local device will immediately start performing with accuracy that is higher than a non-server connected device.

The system can act similarly during analysis of the impact of objective factors on sleep.

Consequently, if the database contains sufficiently large amount of data, collected from users of various types, the process of finding individual sleep characteristics and determination of dependencies of sleep on objective factors for a new user will usually take considerably less time, in such case, compared to local analysis (performed “from scratch”). The remote server will produce results in a few seconds or minutes, while the local device may take days or weeks to collect enough data to get an equivalent quality setting.

FIG. 13C shows a flow chart of this overall server scheme for obtaining, processing, and transmitting sleep related data.

Table 4 gives an analysis of the computational trade-offs for this type of server-based multiple user analysis.

TABLE 4

| Characteristic | Value |
|---|---|
| Resource-intensiveness of | High |
| the algorithm | |
| Algorithm complexity | Relatively high |
| Input data type and means | Similar to the previous method + |
| for its acquisition and storage | All data can be stored on the server side |
| Advantages and | Advantages: Similar to the previous method; |
| disadvantages in comparison | The search for individual sleep characteristics of a |
| with other algorithms | user can be performed considerably faster with the |
| | filled database |
| | Disadvantages: |
| | Similar to the previous method; |
| | Dependency on communication channel with the |
| | server |
| Summary on the efficient | Similar to the previous method; |
| method for algorithm | Necessity of analysis of all available data requires its |
| embodiment. | storage on the server. |
| | There is a need to transfer user's sleep data to the |
| | server and from the server to the device for |
| | awakening. |
| | If needed, some history data can be duplicated on the |
| | client side (device for awakening) |

### Impact of Missing Information:

Although, for optimal performance, users would ideally report feedback on a comprehensive and regular basis, in practice this will not occur. Some users will only provide their general information, i.e. the results of interviewing on global factors, conducted before using the system. Table 5 shows one example of a possible distribution of compliant and non-compliant users.

TABLE 5

| | Group 1 - | Group 2 - | Group 3 - |
|---|---|---|---|
| | enough | not enough | not enough |
| | information | information | information |
| Size of a group (% | 20% | 70% | 10% |
| relatively the | | | |
| general amount of | | | |
| users) | | | |

Information about the user (availability in % relatively the maximum possible)

| | | | |
|---|---|---|---|
| 1. Availability of | 95-100% (the | 40-100% (the | 0-39% (the device |
| daily data on | device is used | device is used | is not used |
| movements during | permanently) | permanently) | permanently) |
| sleep | | | |
| 2. Availability of | 100% (initial | 100% (initial | 100% (initial |
| information on | questionnaire was | questionnaire was | questionnaire was |
| global factors | filled) | filled) | filled) |
| 3. Availability of | 80-100% (the user | 0-10% (the user is | 0-10% (the user is |
| information on | is using the | using the feedback | using the feedback |
| daily factors | feedback | communication | communication |
| | communication | means rarely) | means rarely) |
| | means | | |
| | permanently) | | |
| 4. Availability of | 80-100% (the user | 0-10% (the user is | 0-10% (the user is |
| information on | is using the | using the feedback | using the feedback |
| global factors | feedback | communication | communication |
| change | communication | means rarely) | means rarely) |
| | means | | |
| | permanently) | | |

Depending upon what group level the user is in, the system can perform with varying levels of accuracy. For low frequency users, such as Group 3, the system can simply provide default average values. Group 1 users have provided a lot of data, and thus here the system can generate the most satisfactory results. For Group 2, which will likely be the largest group, some but not all data is available. On the one hand, these users have provided adequate information on their respective global factors, as well as fairly good daily information on movements during sleep. On the other hand, because some daily data is not available, and because some global factors can also change, the system must try to produce the best results possible in view of some loss of data. Group 2 may also include some users that mis-report global factors, such as heavily stressed or alcohol abusing users who are reluctant to admit this problem.

Here, the server can be instructed to make up for the missing information by supplying default information on an as-needed basis. More than one type of default dataset may be stored by the system, and if the results from one default data set receive negative feedback, the system may then attempt to put in the next most likely default data set.

### Data Redundancy Assumption:

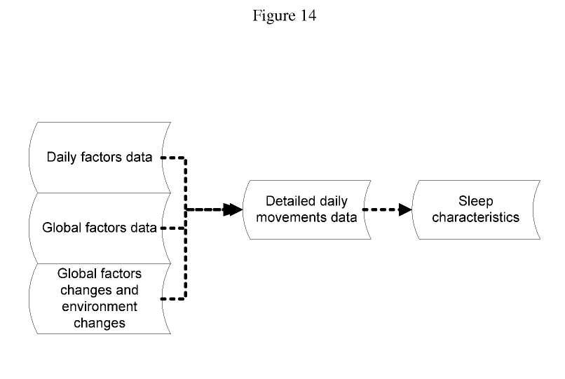

In the case of Group 1, it can be assumed that not only complete information is available, but there is also redundant information on sleep characteristics and their change depending on various factors. That is, there is adequate available information on the objective factors, and their relationship to the user's movement characteristics (and sleep characteristics in general). FIG. 14 shows a figure that represents this interdependence.

In FIG. 14, the “Detailed daily movements data” represents a relatively large amount of daily data on separate user movements during sleep. The “Sleep characteristics” can be considered to be the “combined” descriptive characteristics that are used by the wake-up algorithm.

Since Group 1 represents the most cooperative user group, the system can determine if there is a bidirectional correlation between the “Detailed daily movements' data” and the objective factors. Here such a connection can be assumed to be present; in this case the analysis problem lies in finding the correlation (compliance) between certain characteristics of separate user movements at night, their sequence and occurrence of certain daily factors, and/or change of global factors.

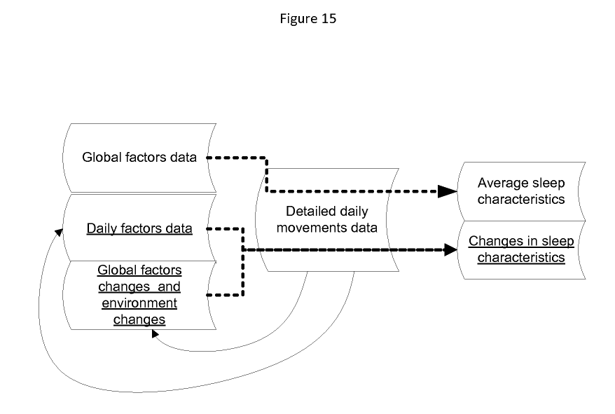

The type of analysis that is possible with the most cooperative group 1 users is shown in FIG. 15. This figure gives an example of the type of analysis that is possible when there is a bidirectional correlation present between the “Detailed daily movements data” on one side, and “Daily factors data” and “Global factors changes” on other side. In this figure, the unknown values for users from group 2 are underlined.

FIG. 15 shows that ideally, finding bidirectional correlation or relationship between a user's detailed daily record of movements during sleep, the user's daily factors, and the user's global movements and environment changes will produce the best results. This is because it is exactly these factors that can cause changes (deviations) of typical sleep characteristics.

Although the quality of the data is not as good, these same considerations, such as the ability to use “Global factors data” are also available for group 2 as well, since the general “Sleep characteristics” for Group 2 are also known. Here the system can attempt to make up for missing data with various default sets of data.

In order to operate the analysis at the highest level of predictive efficiency it is useful to further divide the compliant Group 1 into further subgroups. In these subgroups, “Global factors data”, “Average sleep characteristics” and dependencies between these factors would be expected to tend to be relatively similar for group members within a particular subgroup. Here, with a large user population to draw upon, the server based system can make an even more precise analysis of sleeping patterns.

Here again, a “black box” analysis approach using supervised learning algorithms can be suitable for doing this more precise analysis as well. For example, with using back-propagation neural network algorithms, neural networks can correlate the following data from the “learning” set:

1) as input data—movements during sleep for certain day,
2) as output data—data on factors occurring during certain day.

Basing on the given data, neural network tries to form dependencies between the input and output data for the various subgroups. The overall analysis can be similar to that discussed earlier, but now should be more accurate because it is comparing the user with a more similar group of individuals.

Data compression methods. In some cases, detailed movement data for certain nights may occupy too much storage space in memory for efficient data transmission or storage. In this situation, many methods—i.e. standard lossy and lossless data compression methods, may be used to reduce the amount of data and memory used to store this data.

Analysis of semi-compliant (group 2) users.

Although, for group 2 users, the system will be working with a lesser amount of data, correspondences and patterns previously determined for the group 1 users will presumably continue to be valid. Thus the group 1 rules can be generally applied to the group 2 users as well. In general, the correlation will remain:

Daily factors combination->Change of typical sleep characteristics;
Movements characteristics patterns->Daily factors combination; and
Movements characteristics patterns->Change of typical sleep characteristics, which is generally derived from a combination of the previous two correlations.

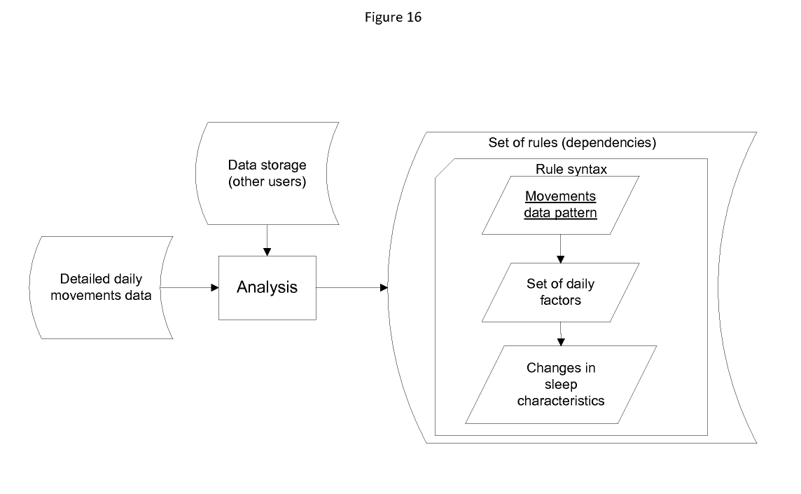

However because some information will be missing, the server system may attempt to compensate for this loss by placing more weight on the analysis of the data it does have, such as the analysis of the user movement patterns at night. This is shown in FIG. 16.

Application of such analysis for the end user from Group 2 would be as follows:

1: To find a corresponding subgroup of Group 1 for the user from Group 2.
2: Identify the availability of movements characteristics patterns of the corresponding subgroup of Group 1 while gathering sleep data of the user from Group 2. These patterns can indicate the availability of certain daily factors with high probability, and sleep characteristics change, consequently. Taking changes in these sleep characteristics into account increases the accuracy of wake-up algorithm.
3: Control check and refinement: If the subsequent determination of sleep characteristics confirms the assumption, we can associate the user with a particular subgroup of Group 1 more efficiently, and apply the rules and dependencies of this group to the user.

### Example 4

Here, sleep movements data for a group 2 user have indicated to the remote server system that the user's falling asleep interval has decreased by 50% (compared to average). According to the previously identified dependencies, such change could be predetermined by the following two complexes of factors:

1) Physical exercises and physical overstrain
or
2) Exhaustion and Lack of sleep quantity and quality for previous days

Here the system can attempt to determine what the most probable factor is. This can be done by measuring movements during the first non-REM interval. If, for example, the movements amplitude has increased, but the frequency has decreased (compared to the average), and, according to pre-identified dependencies, this happens more often in situation 1) than in situation 2) (for example, in case 1) non-REM phase increases by 15% as a rule, and in case 2)—only by 5%), this can be taken into consideration by the remote server and an educated guess as to what is the most suitable wake-up algorithm can be uploaded from the server to the user's local device for awakening.

### Example 5

The system has recorded 5 days of measurements for a group 2 user, and these measurements show a similar significant (or noticeable) deviation of sleep characteristics from normal. However in this case, corresponding typical movement pattern characteristics, which can be associated with particular daily factors, are not found. Note that the system use user's current subgroup's data set in order to find these patterns. This lack of correlation with previously determined situations can indicate that the changes weren't caused by daily (deviating) factors, but rather by other factors such as the change of global factors, changes in sleep environment caused by changing beds (e.g. use of orthopedic mattress) or changes in the user's living environment such as an installation of an air conditioner. In this case the system takes into account this change in the “average” characteristics of the user for a certain period of time (for example 2 weeks), and find another and hopefully more suitable subgroup from Group 1 that again has similar characteristics, and associates the user with this new Group 1 subgroup.

The computational tradeoffs of this type of remote server algorithm are shown in Table 6.

TABLE 6

| Characteristic | Value |
|---|---|
| Resource-intensiveness of | High |
| the algorithm | |
| Algorithm complexity | High |
| Input data type and means | Similar to the previous method |
| for its acquisition and storage | |
| Advantages and | Advantages: Similar to the previous method; |
| disadvantages in comparison | Accuracy of the wake-up algorithm can be increased |
| with other algorithms | even for those users, who don't use feedback |
| | communication means regularly. |
| | Disadvantages: |
| | Similar to the previous method; |
| Summary on the efficient | Similar to the previous method: use of a server is |
| method for algorithm | efficient and necessary |
| embodiment. | |

As can be seen, for this type of data intensive and computationally intensive analysis, use of a server is both efficient and necessary.

The described variants of embodiment and examples were given for better explanation of the useful model and its practical application, and to provide means for understanding the invention by persons of the art. However, the description and the examples herein are for demonstration purposes only. Various modifications and changes are possible within the sense and the formula of the invention.

For example, in some embodiments, it may be useful to produce a lower cost version of the device without a network interface, and that uses pre-programmed sleep phase correction data obtained from a previously generated multiple user supervised learning algorithm.

In other embodiments, again designed for lower cost, it may be useful to allow users to upload their global individual user factors and/or their daily user factors to a remote server analysis system using a different data input and transmission device, such as the user's computer. Here the remote server will analyze the data, and upload the sleep phase correction data back to the alarm clock portion of the device for awakening, but the size or cost of the devices' display screen can be reduced because most of the data entry will be done using the user's computer.

Finally, in a more user friendly if more expensive version, the device for awakening can handle all user data entry and user sleep data display using its own-built in display, and communicate a full set of information (global individual user factors, daily user factors, and measured user movement data) to the remote server, and obtain the most accurate possible sleep phase correction data from the remote server. The remote server can handle many users, continually update its database, and refine its sleep phase correction parameters to higher and higher levels of accuracy; often using supervised learning algorithms.

1. A method for operating a sleep phase alarm clock, said alarm clock comprising a limb mounted motion sensor for monitoring the limb movements of a user during periods of sleep, thus producing measured user movement data, and an alarm clock comprising at least one microprocessor, memory, local software to perform sleep phase analysis of said user, a user interface, and a network connection; said method comprising:
entering in global individual user factors and daily user factors using said user interface;
accepting a wake-up time interval with a beginning time and an end time, and a sleep start time from said user using said user interface;
analyzing said global individual user factors, said daily user factors, and said measured user movement data using said at least one microprocessor, said memory, said local software, and pre-programmed sleep phase correction data, and determining the intersection times between the most probable user REM sleep phase intervals and said wake-up interval;
if said intersection times exist, setting a wake-up time within said intersection times;
if said intersection times do not exist, setting a wake-up time at said end time of said wake-up time interval;
and causing said alarm clock to create a user stimulating signal at said wake-up time.

2. The method of claim 1 in which said global individual user factors are selected from the group consisting of user anthropological data, user lifestyle and schedule data, user sleep environment, user geographical environment, and user general physical state.

3. The method of claim 1, in which said daily user factors are selected from the group consisting of user sports activity, user stress levels, user consumption of pharmaceutically active substances, user medical treatment, user physical or mental overstrain, user food consumption levels, and abnormal user sleep schedules.

4. The method of claim 1, in which said user interface comprises a bit-mapped video display screen, and displaying user interface graphics selected from the group consisting of sleep calendars, column sleep schedule diagrams, and circular sleep schedule diagrams.

5. The method of claim 4, in which the bit-mapped video display screen is a touch sensitive video display screen, and in which said user may input data pertaining to said global individual user factors, said daily user factors, or said wake-up time interval by touching said touch sensitive video display screen.

6. The method of claim 1, in which said pre-programmed sleep phase correction data is generated by a supervised learning algorithm that analyzes said global individual user factors, said daily user factors, and said measured user movement data obtained from a plurality of users.

7. The method of claim 6, further analyzing said user's global individual user factors, said daily user factors, and said measured user movement data, assigning said user to a subgroup, and selecting said pre-programmed sleep phase correction data according to said subgroup.

8. A method for operating a sleep phase alarm clock, said alarm clock comprising a limb mounted motion sensor for monitoring the limb movements of a user during periods of sleep, thus producing measured user movement data, and an alarm clock comprising at least one microprocessor, memory, local software to perform sleep phase analysis of said user, a user interface, and a network connection; said method comprising:
transmitting global individual user factors and daily user factors to a remote network connected server, said remote connected server being connected to a sleep database;
analyzing said global individual user factors and said daily user factors and transmitting sleep phase correction data to said alarm clock using said network connection;
accepting a wake-up time interval with a beginning time and an end time, and a sleep start time from said user using said user interface;
analyzing said measured user movement data using said at least one microprocessor, said memory, said local software, and said sleep phase correction data, and determining the intersection times between the most probable user REM sleep phase intervals and said wake-up interval;
if said intersection times exist, setting a wake-up time within said intersection times;
if said intersection times do not exist, setting a wake-up time at said end time of said wake-up time interval;
and causing said alarm clock to create a user stimulating signal at said wake-up time.

9. The method of claim 8 in which said global individual user factors are selected from the group consisting of user anthropological data, user lifestyle and schedule data, user sleep environment, user geographical environment, and user general physical state.

10. The method of claim 8, in which said daily user factors are selected from the group consisting of user sports activity, user stress levels, user consumption of pharmaceutically active substances, user medical treatment, user physical or mental overstrain, user food consumption levels, and abnormal user sleep schedules.

11. The method of claim 8, in which said user interface comprises a bit-mapped video display screen, and displaying user interface graphics selected from the group consisting of sleep calendars, column sleep schedule diagrams, and circular sleep schedule diagrams.

12. The method of claim 11, in which the bit-mapped video display screen is a touch sensitive video display screen, and in which said user may input data pertaining to said global individual user factors, said daily user factors, or said wake-up time interval by touching said touch sensitive video display screen.

13. The method of claim 8, in which said sleep phase correction data is generated by a supervised learning algorithm that analyzes said global individual user factors, said daily user factors, and said measured user movement data obtained from a plurality of users.

14. The method of claim 13, further analyzing said user's global individual user factors, said daily user factors, and said measured user movement data, assigning said user to a subgroup, and selecting said sleep phase correction data according to said subgroup.

15. The method of claim 8, in which said global individual user factors and daily user factors are entered into a web browser of an independent computerized device and transmitted to said remote network connected server.

16. A method for operating a sleep phase alarm clock, said alarm clock comprising a limb mounted motion sensor for monitoring the limb movements of a user during periods of sleep, thus producing measured user movement data, and an alarm clock comprising at least one microprocessor, memory, local software to perform sleep phase analysis of said user, a user interface, and a network connection; said method comprising:
entering in global individual user factors and daily user factors using said user interface;
using said network connection to transmit said global individual user factors, said daily user factors, and said measured user movement data to a remote network connected server, said remote connected server being connected to a sleep database;
analyzing said global individual user factors, said daily user factors, and said measured user movement data, and transmitting sleep phase correction data to said alarm clock using said network connection;
accepting a wake-up time interval with a beginning time and an end time, and a sleep start time from said user using said user interface;
analyzing said measured user movement data using said at least one microprocessor, said memory, said local software, and said sleep phase correction data, and determining the intersection times between the most probable user REM sleep phase intervals and said wake-up interval;
if said intersection times exist, setting a wake-up time within said intersection times;
if said intersection times do not exist, setting a wake-up time at said end time of said wake-up time interval;
and causing said alarm clock to create a user stimulating signal at said wake-up time.

17. The method of claim 16, in which said method further comprises suggesting to user one or more optimal “go to bed” moments in order to maximize the probability of the intersection of the user REM sleep phase intervals and said wake-up interval.

18. The method of claim 16, in which said sleep phase correction data is generated by a supervised learning algorithm that analyzes said global individual user factors, said daily user factors, and said measured user movement data obtained from a plurality of users.

19. The method of claim 18, in which said supervised learning algorithm is selected from the group consisting of back-propagation artificial neural network algorithms, association rule learning algorithms, and other supervised learning algorithms.

20. The method of claim 18, further analyzing said global individual user factors, said daily user factors, and said measured user movement data, assigning said user to a subgroup, and selecting said sleep phase correction data according to said subgroup.
